(19) United States
(12) Patent Application Publication
(10) Pub. No.: US 2022/0389382 A1
(43) Pub. Date: Dec. 8, 2022

(54) METHOD FOR PREPARING MONOCYTES DERIVED SIGNALING CELLS MIXTURE AND USES THEREOF

(71) Applicant: BLOOMMED TECHNOLOGIES PTE. LTD. (SG), Singapore (SG)

(72) Inventors: YU-CHING HO, New Taipei City (TW); ERIC YI-WEI HU, Underwood Queensland (AU)

(73) Assignee: BLOOMMED TECHNOLOGIES PTE. LTD. (SG), Singapore, SG (SG)

(21) Appl. No.: 17/771,498

(22) PCT Filed: Nov. 3, 2020
(86) PCT No.: PCT/CN2020/126018

§ 371 Date: Apr. 25, 2022

Related U.S. Application Data
(60) Provisional application No. 62/930,534, filed on Nov. 4, 2019.

(51) Int. Cl. C12N 5/0786; A61K 35/15; A61P 1/16

(52) U.S. Cl. CPC C12N 5/0645; A61K 35/15; A61P 1/16; C12N 2500/32; C12N 2500/34; C12N 2500/16; C12N 2500/05

(57) ABSTRACT

A monocytes derived signaling cells mixture is prepared by culturing buffy coats in a cell culture medium containing platelet-poor-plasma and collecting the cultured buffy coats and the cultured cell culture medium. The monocytes derived signaling cells mixture contains granulocytes, lymphocytes, fibrocytes and monocytes, in which the high ratio of the monocytes are CD16 positive monocytes. The monocytes derived signaling cells mixture is used in the improvement of liver function index of a subject.

### CROSS-REFERENCE TO RELATED APPLICATIONS

This application is a national stage application, filed under 35 U.S.C. § 371, of International Patent Application No. PCT/CN2020/126018 filed on Nov. 3, 2020, which claims the benefit of U.S. Provisional Patent Application No. 62/930,534, filed Nov. 4, 2019, each of which is incorporated by reference herein in its entirety.

### FIELD OF THE INVENTION

The present invention relates to a method for preparing a monocytes derived signaling cells mixture by culturing buffy coats in a cell culture medium containing platelet-poor-plasma and collecting the cultured buffy coats and the cultured cell culture medium. The present invention also relates to the monocytes derived signaling cells mixture for the improvement of liver function index in a subject in need of such improvement.

### DESCRIPTION OF PRIOR ART

Liver cancer is the second leading cause of cancer death (8.2% of total cases of cancer death) following lung cancer (18.2%) the first leading cause. There are over 840000 new cases diagnosed in 2018, which is the seventh most commonly occurring cancer for both men and women combined worldwide. The top 10 countries with the highest rates of liver cancer are Mongolia, followed by Egypt, Gambia, Vietnam, Laos, Cambodia, Guinea, Thailand, China, and South Korea according to 2019 report studied by World Cancer Research Fund.

Almost all liver cancer or hepatocellular carcinoma occur in persons with preexisting chronic liver disease, which chronic hepatic inflammation represents an early stage in the carcinogenesis process. In general, chronic inflammation is associated with persistent cell damage and consecutive regeneration, which could lead to fibrosis, cirrhosis, and tumor formation eventually.

Nonsteroidal anti-inflammatory drugs (NSAIDs) is cytokine-based immunotherapies, and mesenchymal stem cells are widely studied and showed beneficial effects in either treating or preventing liver cancer in vitro.

Human circulating monocytes are comprised of a heterogeneous population that can be classified into two main subsets, based on CD14 and CD16 antigens expression. The first group is the CD14+CD16− classical monocytes, making up 80-95% of total circulating monocytes in normal condition, and the second group is CD14±CD16+ nonclassical monocytes, which comprise about 5-20%. Circulating monocytes are mobilized from the bone marrow and recruited to sites of inflammation, where they carry out their respective functions in promoting inflammation or facilitating tissue repair. Circulating monocytes are constantly evolving at the site of inflammation, in which classical monocytes can transition into nonclassical monocytes or differentiate into M1/inflammatory macrophages, dendritic cells or fibrocytes in responding to environmental stimuli and inflammatory signal. CD16+ nonclassical monocytes, so called patrolling monocytes, are able to actively patrol the vascular endothelium to remove damaged cells and debris from the vasculature and have been associated with wound healing and the resolution of inflammation while differentiating into M2 macrophage in damaged tissues.

### SUMMARY

The present invention provides a method for preparing a monocytes derived signaling cells mixture, comprising culturing buffy coats in a cell culture medium containing platelet-poor-plasma; and collecting the cultured buffy coats and the cultured cell culture medium to obtain the monocytes derived signaling cells (MDSC) mixture. The present invention also provides a product of MDSC mixture contains high level of CD 16 positive monocytes and CD34 positive fibrocytes. The present invention further provides a use of a composition for preparing a pharmaceutical composition for improving abnormal liver function index in a subject, wherein the composition comprises a monocytes derived signaling cells (MDSC) mixture.

### BRIEF DESCRIPTION OF THE DRAWINGS

FIG. 1 shows the composition of monocytes pool by CD16- and CD16+ monocytes in ratio. The expression of CD16 antigen of the monocytes derived signaling cells (MDSC)-treated buffy coats were measured at Day 0(D0), Day 2(D2) and Day 4(D4) by flow cytometry.

FIG. 2 shows the population of CD34+ fibrocytes in non-erythrocytes and population changes from Day 0(D0), Day 2(D2) to Day 4(D4) with monocytes derived signaling cells (MDSC) culture treatment in vitro.

### DETAILED DESCRIPTION OF THE INVENTION

Monocytes Derived Signaling Cells (MDSC) culture method results in a compositional change among CD16− and CD16+ monocytes subsets also enhance the differentiation of CD34+ fibrocytes from CD16− monocytes.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The present invention provides a method for preparing a monocytes derived signaling cells mixture, comprising the steps of: (a) centrifuging a peripheral blood from a subject to collect buffy coats and platelet-poor-plasma; (b) mixing the platelet-poor-plasma with a cell nutrients medium to obtain a cell culture medium; (c) culturing the buffy coats in the cell culture medium for about 24-168 hrs; and (d) collecting the cultured buffy coats and the cultured cell culture medium from step (c) to obtain the monocytes derived signaling cells (MDSC) mixture.

In one embodiment, the subject is an animal, preferably a mammal, more preferably a human. In a preferred embodiment, the subject does not suffer from acute viral, bacterial or fungal infection. In a more preferred embodiment, the leukocytes count of the subject is between about 0.5-30×10⁹ nucleated cells/L. In another embodiment, the leukocytes count of the subject is between about 1.8-20×10⁹ nucleated cells/L.

In another embodiment, the step (a) is further divided into step (a1) and step (a2), wherein the step (a1) comprises centrifuging the peripheral blood to separate platelets and leukocytes rich plasma layer and erythrocytes layer, and then collecting the platelets and the leukocytes rich plasma layer; and the step (a2) performed after the step (a1) comprises centrifuging the platelets and leukocytes rich plasma layer to obtain the buffy coats and the platelet-poor-plasma.

As used herein, the term “cell nutrients medium” refers generally to any substance or preparation used for the cultivation of living cells. Any medium suitable for in vitro culturing of mononuclear cells is suitable. A “cell culture” refers to a growth of cells in vitro; although the cells proliferate they do not organize into tissue per se. However, in the specific case of further use of the MDSC mixture for human patients, the cell nutrients medium likes RPMI 1640 medium which is preferably used. In one embodiment, the cell nutrients medium comprises the ingredients of the RPMI 1640 medium. In a preferred embodiment, the ingredients of the cell nutrients medium comprise but not limited to amino acids, glucose, sodium di-H phosphate, calcium chloride, magnesium chloride, potassium acetate, sodium chloride and dihydrogen monoxide.

In one embodiment, the volume ratio of the platelet-poor-plasma and the cell nutrients medium is 10:1 to 1:1. In a preferred embodiment, the volume ratio of the platelet-poor-plasma and the cell nutrients medium is 5:1 to 1:1. In a more preferred embodiment, the volume ratio of the platelet-poor-plasma and the cell nutrients medium is 2:1 to 1:1. In another embodiment, the volume ratio of the platelet-poor-plasma and the cell nutrients medium is 2:1.

In another embodiment, the cell density of the buffy coats ranges from about 2.8-100×10⁹ nucleated cells/L. In a preferred embodiment, the cell density of the buffy coats ranges from about 5.6-68×10⁹ nucleated cells/L. In a more preferred embodiment, the cell density of the buffy coats ranges from about 9.6-36×10⁹ nucleated cells/L.

In one embodiment, the buffy coats comprise monocytes, fibrocytes, lymphocytes and granulocytes. In a preferred embodiment, the cell ratio of the buffy coats is about 1-14% monocytes, about 0.1-5% fibrocyte, about 20-80% lymphocytes and about 10-70% granulocytes.

The buffy coats and the cell culture medium are incubated at 37° C. with 5% CO₂ for 24-168 hrs, and then are collected to form the MDSC mixture after incubation. Therefore, the components of the MDSC mixture comprise the buffy coats and the cell culture medium after incubation. In one embodiment, the time for culturing the buffy coats in the cell culture medium for about 24-120 hrs. In a preferred embodiment, the time for culturing the buffy coats in the cell culture medium for about 48-96 hrs. In a more preferred embodiment, the time for culturing the buffy coats in the cell culture medium for about 96 hrs.

In the present invention, the monocytes derived signaling cells (MDSC) is a mixture of leukocytes, consisting of classical, intermediate, non-classical monocytes and fibrocytes as major active components.

In one embodiment, the cell density of the MDSC mixture ranges from about 0.35-10×10⁹ nucleated cells/L. In a preferred embodiment, the cell density of the MDSC mixture ranges from about 0.7-6.8×10⁹ nucleated cells/L. In a more preferred embodiment, the cell density of the MDSC mixture ranges from about 1.2-3.6×10⁹ nucleated cells/L.

The monocytes derived signaling cells (MDSC) mixture of the present invention can be further concentrated by removing the supernatant liquid of the MDSC mixture or diluted with 0.9% saline to adjust cell density. In another embodiment, the method for preparing the MDSC mixture further comprises the step (e), after the step (d), for adjusting the cell density of the MDSC mixture by removing the supernatant liquid of the MDSC mixture or diluting with saline.

In one embodiment, the MDSC mixture comprises granulocytes, lymphocytes, fibrocytes and monocytes. In a preferred embodiment, the cell ratio of the MDSC mixture comprises about 25-80% granulocytes, about 15-70% lymphocytes, about 0.3-15% fibrocytes and about 0.5-15% monocytes. In another embodiment, the cell ratio of the MDSC mixture comprises about 0.3-15% CD34+ fibrocytes. When the buffy coats are cultured in the cell culture medium about 96 (±12) hrs, the cell ratio of the fibrocytes in the MDSC mixture is about 2-6%. In a preferred embodiment, the cell ratio of the MDSC mixture comprises about 2-6% CD34+ fibrocytes.

In another embodiment, the cell ratio of the monocytes comprises about 1-25% CD16− monocytes and about 75-99% CD16+ monocytes. In a preferred embodiment, the cell ratio of the monocytes comprises about 2-7% CD16− monocytes and about 93-98% CD16+ monocytes.

The cells of the buffy coats will secrete various cytokines during the MDSC culturing period. In one embodiment, the cytokines of the MDSC mixture comprise angiogenin, epidermal growth factor (EGF), neutrophil activating peptide 2 (NAP-2), leptin, platelet-derived growth factor-BB (PDGF-BB), Regulated on Activation, Normal T Cell Expressed and Secreted (RANTE), adipocyte complement-related protein of 30 kDa (Acrp 30), interleukin-6 receptor (IL-6R), tissue inhibitor of metalloproteinases 2 (TIMP-2), membrane scaffold protein-a (MSP-a), monocyte chemoattractant protein-1 (MCP-1), LIGHT (known as tumor necrosis factor superfamily member 14 (TNFSF14)), stromal cell-derived factor 1 (SDF-1), IL-8, thrombopoietin (TPO), vascular endothelial growth factor (VEGF), brain-derived neurotrophic factor (BDNF), IL-10, insulin-like growth factor-1 (IGF-1), transforming growth factor-beta (TGF-β), neutrophil activating peptide 2 (NAP-2), stem cell factor (SCF), intercellular adhesion molecule 1 (ICAM-1) and urokinase-type plasminogen activator receptor (uPAR).

In one embodiment, the method for preparing the MDSC mixture further comprises a step (f), after the step (d), for removing the cells from the MDSC mixture to obtain a MDSC conditioned medium. Therefore, the MDSC conditioned medium does not contain any cells.

In one embodiment, the MDSC conditioned medium comprises but not limited to amino acids, glucose, sodium di-H phosphate, calcium chloride, magnesium chloride, potassium acetate, sodium chloride and dihydrogen monoxide. In another embodiment, the MDSC conditioned medium comprises but not limited to angiogenin, EGF, NAP-2, leptin, PDGF-BB, RANTE, Acrp 30, IL-6R, TIMP-2, MSP-a, MCP-1, LIGHT, SDF-1, IL-8, TPO, VEGF, BDNF, IL-10, IGF-1, TGF-b, NAP-2, SCF, ICAM-1 and uPAR.

The present invention also provides a product of monocytes derived signaling cells (MDSC) mixture, wherein the cell ratio of the MDSC mixture comprises 25-80% granulocytes, 15-70% lymphocytes, 0.3-15% fibrocytes and 0.5-15% monocytes, wherein the cell ratio of the monocytes comprises 75-99% CD16+ monocytes. The MDSC mixture is obtained by a method for culturing buffy coats, comprising the steps of: (a) centrifuging a peripheral blood from a subject to collect buffy coats and platelet-poor-plasma; (b) mixing the platelet-poor-plasma with a cell nutrients medium to obtain a cell culture medium; (c) culturing the buffy coats in the cell culture medium for about 24-168 hrs; and (d) collecting the cultured buffy coats and the cultured cell culture medium from step (c) to obtain the monocytes derived signaling cells (MDSC) mixture.

In one embodiment, the subject is a human with leukocytes count between about 0.5-30×10⁹ nucleated cells/L. In a preferred embodiment, the subject is a human with leukocytes count between about 1.8-20×10⁹ nucleated cells/L.

In one embodiment, the cell density of the MDSC mixture ranges from about 0.35-10×10⁹ nucleated cells/L. In a preferred embodiment, the cell density of the MDSC mixture ranges from about 0.7-6.8×10⁹ nucleated cells/L. In a more preferred embodiment, the cell density of the MDSC mixture ranges from about 1.2-3.6×10⁹ nucleated cells/L.

In one embodiment, the MDSC mixture comprises granulocytes, lymphocytes, fibrocytes and monocytes. In a preferred embodiment, the cell ratio of the MDSC mixture comprises about 25-80% granulocytes, about 15-70% lymphocytes, about 0.3-15% fibrocytes and about 0.5-15% monocytes. In another embodiment, the cell ratio of the MDSC mixture comprises about 0.3-15% CD34+ fibrocytes. In a preferred embodiment, the cell ratio of the MDSC mixture comprises about 2-6% CD34+ fibrocytes.

In another embodiment, the cell ratio of the monocytes comprises about 1-25% CD16− monocytes and about 75-99% CD16+ monocytes. In a preferred embodiment, the cell ratio of the monocytes comprises about 2-7% CD16− monocytes and about 93-98% CD16+ monocytes.

In one embodiment, the ingredients of the MDSC mixture comprise but not limited to amino acids, glucose, sodium di-H phosphate, calcium chloride, magnesium chloride, potassium acetate, sodium chloride and dihydrogen monoxide. In another embodiment, the MDSC mixture comprises but not limited to angiogenin, EGF, NAP-2, Leptin, PDGF-BB, RANTE, Acrp 30, IL-6R, TIMP-2, MSP-a, MCP-1, LIGHT, SDF-1, IL-8, TPO, VEGF, BDNF, IL-10, IGF-1, TGF-b, NAP-2, SCF, ICAM-1 and uPAR.

The present invention also provides a composition comprising a cell nutrients medium and a plasma.

In one embodiment, the cell nutrients medium comprises the ingredients of the RPMI 1640 medium. In a preferred embodiment, the ingredients of the cell nutrients medium comprise but not limited to amino acids, glucose, sodium di-H phosphate, calcium chloride, magnesium chloride, potassium acetate, sodium chloride and dihydrogen monoxide.

In another embodiment, the plasma is platelet-poor-plasma. In one embodiment, the plasma is obtained from a subject. In a preferred embodiment, the subject is a human. In a more preferred embodiment, the subject is a human with leukocytes count between about 0.5-30×10⁹ nucleated cells/L. In another embodiment, the subject is a human with leukocytes count between about 1.8-20×10⁹ nucleated cells/L.

The present invention further provides a method for improving abnormal liver function index in a subject with abnormal liver function index, comprising administering to the subject with abnormal liver function index an effective amount of composition comprising a monocytes derived signaling cells (MDSC) mixture, wherein the cell ratio of the MDSC mixture comprises 25-80% granulocytes, 15-70% lymphocytes, 0.3-15% fibrocytes and 0.5-15% monocytes, wherein the cell ratio of the monocytes comprises 75-99% CD16+ monocytes. The MDSC mixture is obtained by a method for culturing buffy coats, comprising the steps of: (a) centrifuging a peripheral blood from the subject with abnormal liver function index to collect buffy coats and platelet-poor-plasma; (b) mixing the platelet-poor-plasma with a cell nutrients medium to obtain a cell culture medium; (c) culturing the buffy coats in the cell culture medium for about 24-168 hrs; and (d) collecting the cultured buffy coats and the cultured cell culture medium from the step (c) to obtain the monocytes derived signaling cells (MDSC) mixture.

In one embodiment, the subject is a human. In a preferred embodiment, the subject does not suffer from acute viral, bacterial or fungal infection. Therefore, the MDSC mixture is used in the subject who provides the peripheral blood in self for culturing.

In another embodiment, the subject is a human with leukocytes count between about 0.5-30×10⁹ nucleated cells/L. In a preferred embodiment, the subject is a human with leukocytes count between about 1.8-20×10⁹ nucleated cells/L.

The term “administration” or “administering” is used throughout the specification to describe the process by which the MDSC mixture according to the present invention are delivered to the subject. The MDSC mixture of the present invention is administered to the subject through a number of different routes known in the art. In one embodiment, the MDSC mixture can be applied via topical, subcutaneous, intravenous or intraarticular administration. In a preferred embodiment, the administration of the MDSC mixture comprises intravenous administration.

In certain embodiments, the subject is in need of improved liver function index. “Improves liver function index” means to change liver function index toward normal limits. In certain embodiments, improved liver function index is measured by a reduction in the value of the glutamic oxaloacetic transaminase (GOT), glutamic pyruvic transaminase (GPT), total protein, albumin, globulin, alkaline phosphatase (ALP), total bilirubin, gamma-glutamyl transferase (GGT), triglyceride and cholesterol, etc. In another embodiment, the liver function index comprises GOT, GPT, total protein, albumin, globulin, ALP, total bilirubin, GGT, triglyceride and cholesterol. In a preferred embodiment, the liver function index comprises GOT, GPT, total protein, albumin, ALP, GGT, triglyceride and cholesterol.

In one embodiment, the cell density of the MDSC mixture ranges from about 0.35-10×10⁹ nucleated cells/L. In a preferred embodiment, the cell density of the MDSC mixture ranges from about 0.7-6.8×10⁹ nucleated cells/L. In a more preferred embodiment, the cell density of the MDSC mixture ranges from about 1.2-3.6×10⁹ nucleated cells/L.

In one embodiment, the MDSC mixture comprises granulocytes, lymphocytes, fibrocytes and monocytes. In a preferred embodiment, the cell ratio of the MDSC mixture comprises about 0.5-15% monocytes. In another embodiment, the cell ratio of the MDSC mixture comprises about 0.3-15% CD34+ fibrocytes. In a preferred embodiment, the cell ratio of the MDSC mixture comprises about 2-6% CD34+ fibrocytes.

In another embodiment, the cell ratio of the monocytes comprises about 1-25% CD16− monocytes and about 75-99% CD16+ monocytes. In a preferred embodiment, the cell ratio of the monocytes comprises about 2-7% CD16− monocytes and about 93-98% CD16+ monocytes.

In one embodiment, the ingredients of the MDSC mixture comprise but not limited to amino acids, glucose, sodium di-H phosphate, calcium chloride, magnesium chloride, potassium acetate, sodium chloride and dihydrogen monoxide. In another embodiment, the MDSC mixture comprises but not limited to angiogenin, EGF, NAP-2, leptin, PDGF-BB, RANTE, Acrp 30, IL-6R, TIMP-2, MSP-a, MCP-1, LIGHT, SDF-1, IL-8, TPO, VEGF, BDNF, IL-10, IGF-1, TGF-b, NAP-2, SCF, ICAM-1 and uPAR.

The present invention also provide a use of a composition for preparing a pharmaceutical composition for improving abnormal liver function index, wherein the composition comprises a monocytes derived signaling cells (MDSC) mixture, wherein the cell ratio of the MDSC mixture comprises 25-80% granulocytes, 15-70% lymphocytes, 0.3-15% fibrocytes and 0.5-15% monocytes, wherein the cell ratio of the monocytes comprise 75-99% CD16+ monocytes. The MDSC mixture is obtained by a method for culturing buffy coats, comprising the steps of: (a) centrifuging a peripheral blood from a subject with abnormal liver function index to collect buffy coats and platelet-poor-plasma; (b) mixing the platelet-poor-plasma with a cell nutrients medium to obtain a cell culture medium; (c) culturing the buffy coats in the cell culture medium for about 24-168 hrs; and (d) collecting the cultured buffy coats and the cultured cell culture medium from the step (c) to obtain the monocytes derived signaling cells (MDSC) mixture.

In one embodiment, the subject is a human with leukocytes count between about 0.5-30×10⁹ nucleated cells/L. In a preferred embodiment, the subject is a human with leukocytes count between about 1.8-20×10⁹ nucleated cells/L.

In one embodiment, the administration of the pharmaceutical composition comprises intravenous administration.

In another embodiment, the liver function index comprises GOT, GPT, total protein, albumin, globulin, ALP, total bilirubin, GGT, triglyceride and cholesterol. In a preferred embodiment, the liver function index comprises GOT, GPT, total protein, albumin, ALP, GGT, triglyceride and cholesterol. The MDSC mixture of the present invention can lower the value of GOP, GPT and GGT for improving abnormal liver function index.

In one embodiment, the cell density of the MDSC mixture ranges from about 0.35-10×10⁹ nucleated cells/L. In a preferred embodiment, the cell density of the MDSC mixture ranges from about 0.7-6.8×10⁹ nucleated cells/L. In a more preferred embodiment, the cell density of the MDSC mixture ranges from about 1.2-3.6×10⁹ nucleated cells/L.

In one embodiment, the MDSC mixture comprises granulocytes, lymphocytes, fibrocytes and monocytes. In a preferred embodiment, the cell ratio of the MDSC mixture comprises about 25-80% granulocytes, about 15-70% lymphocytes, about 0.3-15% fibrocytes and about 0.5-15% monocytes. In another embodiment, the cell ratio of the MDSC mixture comprises about 0.3-15% CD34+ fibrocytes. In a preferred embodiment, the cell ratio of the MDSC mixture comprises about 2-6.0% CD34+ fibrocytes.

In another embodiment, the cell ratio of the monocytes comprises about 1-25% CD16− monocytes and about 75-99% CD16+ monocytes. In a preferred embodiment, the cell ratio of the monocytes comprises about 2-7% CD16− monocytes and about 93-98% CD16+ monocytes.

In one embodiment, the MDSC mixture comprises but not limited to amino acids, glucose, sodium di-H phosphate, calcium chloride, magnesium chloride, potassium acetate, sodium chloride, dihydrogen monoxide, angiogenin, EGF, NAP-2, Leptin, PDGF-BB, RANTE, Acrp 30, IL-6R, TIMP-2, MSP-a, MCP-1, LIGHT, SDF-1, IL-8, TPO, VEGF, BDNF, IL-10, IGF-1, TGF-b, NAP-2, SCF, ICAM-1 and uPAR.

### Examples

The present invention may be implemented in many different forms and should not be construed as limited to the examples set forth herein. The described examples are not limited to the scope of the present invention as described in the claims.

Materials and Methods:

Peripheral Blood Collection

Individual with leukocytes count between 1.8-20×10⁹ nucleated cells/L measured by clinical laboratory complete blood count (CBC) testing and with no acute viral, bacterial and fungal infection, such as human immunodeficiency virus (HIV), syphilis and active hepatitis B virus (HBV) or sepsis is subjected to the following procedure. Standard peripheral blood draw with strict aseptic technique was performed to avoid contaminating sample culture with skin organism and needle size 16-18 gauge was used to avoid blood sample hemolysis. Peripheral blood was collected using triple blood bags, mixed with 0.14 (ml) anticoagulant per milliliter in primary bag and stored in 4-6° C. before lab processing.

Laboratory Processing

(1) Buffy Coat Isolation

Buffy coats were isolated from anticoagulated peripheral blood by gradient centrifugation following blood banking protocol using refrigerated floor standing centrifuge (Sorvall XFR, Thermo Fisher). Two spins were performed to obtain the platelet-poor-plasma and buffy coats. Whole blood was separated into platelets and leukocytes rich plasma layer and erythrocytes layer by first centrifugation at speed 1800×g for 3-5 mins. Platelets and leukocytes rich plasma layer was identified by visual observation of yellow-orangish color and transferred into first satellite bag using component extractor. Platelets and leukocytes layer then was spun at speed 2500×g for 8-10 mins to separate buffy coats layer and platelet-poor-plasma layer. Platelet-poor-plasma layer was identified by visual observation of yellowish color and transferred into second satellite bag for culture medium preparation. Buffy coats layer was left in the first satellite bag for cell culture sample preparation.

(2) Culture Medium Preparation

The cell culture medium for preparing the monocytes derived signaling cells (MDSC) mixture was prepared with human platelet-poor-plasma and cell nutrients solution in a ratio of 2:1. The cell nutrients solution was prepared in the similar formulation as Roswell Park Memorial Institute (RPMI) 1640 medium. The ingredients of the cell nutrients solution included amino acids, glucose, sodium di-H phosphate, calcium chloride, magnesium chloride, potassium acetate, sodium chloride and dihydrogen monoxide. Human plate-poor-plasma was prepared from peripheral blood by gradient centrifugation from the same sample as the one used for buffy coat collection.

(3) Cell Culture Sample Preparation

The cell density of the collected buffy coats was about 2.8-100×10⁹ nucleated cells/L. The nucleated cells of the collected buffy coats comprised about 1-14% monocytes, 0.1-5% fibrocyte, 20-80% lymphocytes and 10-70% granulocytes. Cell culture sample was prepared by mixing buffy coats and the cell culture medium. Buffy coats were resuspended into the cell culture medium to achieve cell density of 1.2-3.6×10⁹ nucleated cells/L. The cell culture sample then was sampled for total nucleated cell (TNC) count using automated hematology analyzer and transferred into cell culture vessel, which was highly air permeable and for suspension culture.

(4) Incubation

The cell culture sample was incubated at 37° C. with 5% CO₂ for 24-120 hrs before harvest. The cell culture sample was sampled before and after incubation to observe the changes of cell phenotype and supernatant profile along the cultivation.

(5) Harvest

The cell culture sample was harvested after 24 to 120 hours incubation. Mechanical vibration was used to detach cells from the base, then cells were resuspended into culture supernatant. Cultured cells (buffy coats) mixed with culture supernatant (cultured cell culture medium) were together integrated into MDSC final product.

(6) MDSC

MDSC final products contained about 1.2-3.6×10⁹ nucleated cells/L; 25-80% granulocyte, 15-70% lymphocyte, 0.3-15% fibrocyte and 0.5-15% monocyte (monocytes subsets: 1-25% CD16− monocytes and 75-99% CD16+ monocytes). Other components of the MDSC final product included amino acid, glucose, sodium di-h phosphate, calcium chloride, magnesium chloride, potassium acetate, sodium chloride and dihydrogen monoxide, presenting as excipient for the function of cell nutrient, tonicity adjustment, diluent/stabilize and pH buffer in the MDSC final product.

The MDSC final product required 4-6° C. storage condition to keep cells decline rate around 0.5-1.0% per hour and should not be administered 24 hours after cell harvest.

Biological Study

The cell culture samples were sampled before and after incubation for sample analysis. Cells' phenotype, viability and supernatant profile were analyzed by flow cytometer, automated hematology analyzer and cytokine array following manufacture's protocol.

(a) TNC Count

Total nucleated cell (TNC) counts were determined with automated hematology analyzer (Sysmex XN-2000, Sysmex) using direct current (DC) sheath flow detection method and multi-angle polarized scatter separation (MAPSS) technique to provide the primary TNC count, the sum of white blood cell (WBC) and nucleated red blood cell (NRBC). The TNC was calculated by multiplying the TNC for the total volume of the sample.

(b) Flow Cytometric Analysis

Flow cytometer (FacsCanto II Flow Cytometer, BD Bioscience) was used for detecting and measuring the cluster of differentiation (CD) antigen expression of cells labeling with fluorescent marker.

Monocyte subsets were analyzed with flow cytometric immunophenotyping method using monoclonal antibodies anti-human CD45+ fluorescein isothiocyanate (FITC, BioLegend), 7-amino-actinomycin (7-AAD viability staining, BioLegend), anti-human CD14+ allophycocyanine (APC, BioLegend) and anti-human CD16+ fluorescein isothiocyanate (FITC, BioLegend). The monoclonal antibodies anti-human CD45+ fluorescein isothiocyanate (FITC, BioLegend), 7-amino-actinomycin (7-AAD viability staining, BioLegend), anti-human CD14+ allophycocyanine (APC, BioLegend) and anti-human CD34+ fluorescein isothiocyanate (FITC, BioLegend) were used for circulating fibrocytes analysis.

(c) Composition of Monocytes Pool Analysis

Human monocytes were recognized as a heterogeneous population that could be classified into three subsets based on its CD14 and CD16 antigen expression. These populations including CD14+CD16− monocyte (known as classical monocyte), CD14+CD16+ monocyte (intermediate), and CD14-CD16+ monocyte (non-classical) were phenotypically and functionally different.

The composition of monocytes subsets were analyzed with flow cytometric immunophenotyping results and TNC count to calculate the absolute number of each monocyte subset, then were divided by the total monocytes count to get the composition of monocytes subsets. Total monocytes count was decided by the percentage of monocytes in total leukocytes population and TNC count. The percentage of monocytes in total leukocytes population was decided according to the cell size, granularity and expression of CD14/CD16 markers on cell surface.

Samples were measured and analyzed at the point before incubation, 48 hrs incubation and 96 hrs incubation to observe the change of monocytes subsets composition throughout the MDSC cell culture period.

(d) Population of Fibrocytes Analysis

Circulating fibrocytes were a leukocyte subpopulation, characterized by cellular phenotype CD45+CD34+ using flow cytometry analysis. The population of fibrocytes were calculated with the number of CD34 positive cells divided by the total number of viable CD45 positive cells within the gating of the monocyte population. Samples were measured and analyzed at the point before incubation, 48 hrs incubation and 96 hrs incubation to observe the population change of fibrocytes throughout the MDSC cell culture period.

(e) Cytokine Array

Human plasma contained various cytokines/chemokines and growth factors. The MDSC culture method could enhance the secretion of certain types of cytokines and growth factors, such as IL-10, TGF-beta, VEGF and Angiogenin . . . etc.

Human cytokines in MDSC culture supernatants from sample before incubation and sample at 96 hrs incubation were identified and measured with sandwich immunoassay (ELISA) method using cytokine array (Human Cytokine Antibody Array 6 & 7, RayBiotech) following manufacturer's protocol. The Human Cytokine Array Kit consists of a nitrocellulose membrane containing total 120 different anti-cytokines/chemokines antibodies spotted in duplicate. The 120 cytokines/chemokines included angiopoietin (ANG), brain-derived neurotrophic factor (BDNF), bone morphogenetic protein 4 (BMP4), BMP6, chemoattractant cytokine ligand 1 (CCL1), CCL11, CCL13, CCL15, CCL17, CCL18, CCL2, CCL20, CCL22, CCL23, CCL24, CCL26, CCL5, CCL7, CCL8, cilinary neuronotrophic factor (CNTF), colony-stimulating factor 1 (CSF1), CSF2, CX3CL1, CXCL12, CXCL13, CXCL6, CXCL9, epidermal growth factor (EGF), fibroblast growth factor 6 (FGF6), FGF7, Fms related receptor tyrosine kinase 3 ligand (FLT3LG), glial cell-derived neurotrophic factor (GDNF), interferon gamma (IFNγ), insulin-like growth factor 1 (IGF1), insulin-like growth factor-binding protein 1 (IGFBP1), IGFBP2, IGFBP4, IL10, IL13, IL15, IL16, IL1A, IL1B, IL1RN, IL2, IL3, IL4, IL5, IL6, IL7, KIT Ligand (KITLG), leptin (LEP), lipoteichoic acid (LTA), neurotrophin 3 (NTF3), platelet derived growth factor subunit B (PDGFβ), Pro-platelet basic protein (PPBP), transforming growth factor beta 1 (TGFβ1), TGFB3, tumor necrosis factor (TNF), tumor necrosis factor superfamily member 14 (TNFSF14), adiponectin (ADIPOQ), agouti-related protein (AGRP), angiopoietin 2 (ANGPT2), amphiregulin (AREG), AXL, betacellulin (BTC), CCL16, CCL19, CCL25, CCL27, CCL28, CCL3, CCL4, colony stimulating factor 3 (CSF3), CXCL1, CXCL11, CXCL2, CXCL3, CXCL5, CXCL8, epidermal growth factor receptor (EGFR), FAS, fibroblast growth factor 2 (FGF2), FGF4, FGF9, gastric intrinsic factor (FIGF), human growth hormone (HGF), intercellular adhesion molecule 1 (ICAM1), ICAM3, insulin like growth factor 1 receptor (IGF1R), IGFBP3, IGFBP6, IL11, IL12A, IL12B, IL17A, IL1R1, IL1RL1, IL2RA, IL6R, IL6ST, macrophage migration inhibitory factor (MIF), macrophage stimulating 1 (MST1), nerve growth factor (NGF), neurotrophin 4 (NTF4), oncostatin M (OSM), placenta growth factor (PGF), plasminogen activator urokinase receptor (PLAUR), thrombopoietin (THPO), tissue inhibitors of metalloproteinases (TIMP1), TIMP2, tumor necrosis factor receptor superfamily member 10C (TNFRSF10C), TNFRSF10D, TNFRSF11B, TNFRSF18, TNFRSF1A, TNFRSF1B, TNFSF18, thrombopoietin (TPO), TYRO3, vascular endothelial growth factor A (VEGFA) and X-C Motif Chemokine Ligand 1 (XCL1).

The results were measured with chemiluminescent detection system and analyzed with analysis software tool (The Q-Analyzer, RayBiotech). The data were expressed as the change in expression of cytokines/chemokines of MDSC mixture after 96-hrs-incubation comparing to MDSC mixture before incubation.

Results:

(i) Transition of Monocytes Pool with MDSC Culture Treatment In Vitro

Circulating monocytes were a heterogenous group comprised of CD16− monocytes for 80-95% population and CD16+ monocytes for 5-20% in human peripheral blood.

According to MDSC culture treatment study, the composition of monocytes pool changed significantly 48 hours after buffy coats treated by MDSC culture method comparing to the sample at Day 0 (before incubation) and continued the observation till 96 hours. Samples were analyzed every 48 hrs from Day 0, Day 2 and Day 4 with flow cytometric immunophenotyping method to understand the transition of monocytes pool composition.

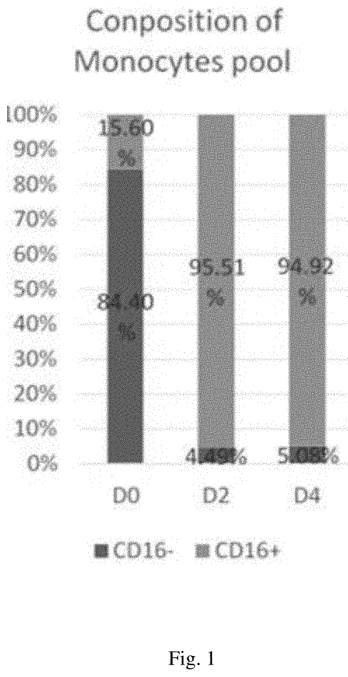

CD16− monocytes was showing as dominant group of the Day 0 sample that composing an 84.40% of total monocytes population and CD16+ monocytes for 15.6% in average, which gave the ratio of CD16− to CD16+ about 17:3. At Day 2 analysis, proportion of CD16− monocytes to CD16+ monocytes was about 1:19, that CD16− decreased significantly from 84.4% to 4.49%, in contrast, CD16+ monocytes increased from 15.6% to 95.51%. And the ratio kept similar from Day 2 to Day 4 that CD16− monocytes composed of 5.08% of total monocytes population and CD16+ monocytes for 94.92% of total monocytes population, ratio 1:19 (see FIG. 1).

(ii) Induction of CD34 Antigen Expression on Monocytes with MDSC Culture Treatment In Vitro

CD34+ fibrocytes derived from CD16− circulating monocytes comprised about 0.1-1.0% of circulating non-erythrocytic cells in peripheral blood.

According to MDSC culture treatment study, the population of CD34+ fibrocytes increased significantly 96 hours after buffy coats treated with MDSC culture method comparing to the sample at Day 0 (before incubation). Samples were analyzed every 48 hrs from Day 0, Day 2 and Day 4 with flow cytometric phenotyping method to understand the population change of CD34+ fibrocytes in non-erythrocytic cells with MDSC culture method in vitro.

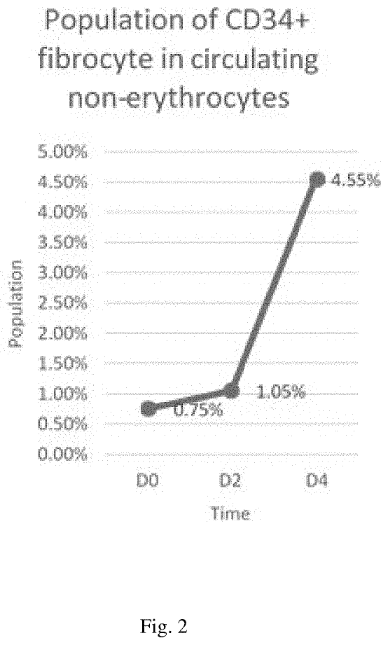

Basing on the cell immunophenotype testing result, CD34+ fibrocytes comprised about 0.75% of total non-erythrocytic cell in the sample treated with MDSC culture method at Day 0 in average. The population of CD34+ fibrocytes increase to 1.05% at Day 2, which was about +0.3% population change. A six-fold increase of CD34+ fibrocytes population was observed at Day 4 compared to Day 0, that the population of CD34+ fibrocytes grew from 0.75% at Day 0 to 4.55% at Day 4 (see FIG. 2).

(iii) Cytokines Profile of Buffy Coats Treated with MDSC Culture Method

A total 120 different kinds of human cytokines (including chemokines and growth factors) array kit was used to analyze the cytokines profile of MDSC-treated buffy coats cultures. Samples were analyzed at Day 0 (before incubation) and Day 4 (after incubation).

According to the test results, 22 cytokines showed high-level expression with visible spots on the cytokine array image. High-level expressed cytokines included angiogenin, EGF, NAP2, leptin, PDGF-BB, RANTE, Acrp 30, IL-6R, TIMP-2 and MSP-a etc.

According to analysis results, 8 cytokines showed a significant increase between Day 0 and Day 4, which cytokine level of Day 4 had more than 100% increasement comparing to Day 0, including MCP-1, TARC, IL-8, MSP-a, TECK, TPO, VEGF and VEGF-D (see Table 1).

TABLE 1
List of cytokines increased more than 100%
in samples treated with MDSC culture method

| | Sample 1 | Sample 2 | |
|---|---|---|---|
| Cytokines | (D 0) | (D 4) | Sample 2/Sample 1 |
| MCP-1 | 63038 | 155645 | 246.91% |
| TARC | 40213.6 | 132430 | 329.32% |
| IL-8 | 66716 | 460695 | 690.53% |
| MSP-a | 67305 | 371170 | 551.47% |
| TECK | 10665.9 | 22548.65 | 211.41% |
| TPO | 125300 | 436135 | 348.07% |
| VEGF | 8092 | 51806 | 640.23% |
| VEGF-D | 30576 | 67256 | 219.97% |

Cases Study 1. Liver Function Index Improvement

In verifying the treating concept of MDSC in the improvement of liver function index, clinical cases treated with single dose MDSC cellular product through standard intravenous (IV) infusion was reviewed and presented in the following content. General liver function and lipid profile were monitored before and after MDSC treatment to evaluate the treatment safety and efficacy. The liver function index comprised glutamic oxaloacetic transaminase (GOT), glutamic pyruvic transaminase (GPT), total protein, albumin, globulin, alkaline phosphatase (ALP), total bilirubin, gamma-glutamyl transferase (GGT), triglyceride and cholesterol.

Four cases of non-viral hepatitis patients with GOT and/or GPT abnormal were studied to verify the liver-function-improving effect of MDSC. Case 1 showed 23.81% decrease of GPT, 32.65% decrease of GGT and 23.68% decrease of cholesterol level in 1.5 months. No eating restriction and exercise adjustment were practiced during the post-treatment period. In Case 2, GOT and GPT were decreased by 21.74% and 29.73% in 3.5 months. In Case 3, GOP, GPT and GGT were improved with 37.14%, 17.86% and 36% drop in 15 days. Case 4 showed a significant improvement of both GOT and GPT by more than 60% decline in 6 months. Based on the treatment results of these four cases, the decreases of abnormal liver function index (comprising GOT, GPT, total protein, albumin, ALP, GGT, triglyceride and cholesterol) indicated the treating effect of the MDSC.

Case 1. Age 29, Male, Chief complaint: food allergy
with skin rush, Treatment date: 17 Jan. 2019

| | Before | 1.5 months | | |
|---|---|---|---|---|
| | treatment Date: | post Date: | Normal | |
| Test/Date | 14 Jan. 2019 | 4 Mar. 2019 | Range | Unit |
| GOT/ALT | 73 | 72 | 0-45 | U/L |
| GPT/AST | 42 | 32 | 0-41 | U/L |
| Total Protein | 84 | 75 | 60-82 | g/L |
| Albumin | 52 | 44 | 35-50 | g/L |
| Globulin | 32 | 31 | 20-40 | g/L |
| Alkaline | 78 | 72 | 30-115 | U/L |
| Phosphatase | | | | |
| (ALP) | | | | |
| Total Bilirubin | 18 | 15 | 2-20 | umol/L |
| GGT | 49 | 33 | 0-70 | U/L |
| Triglyceride | 2.8 | 2.5 | 0.3-4.0 | mmol/L |
| Total | 7.6 | 5.8 | 3.6-7.3 | mmol/L |
| Cholesterol | | | | |

Case 2. Age 62, Male, Chief complaint: metabolic
disease, Treatment date: Feb. 22, 2019

| | Before | 3.5 months | | |
|---|---|---|---|---|
| | treatment Date: | post Date: | Normal | |
| Test/Date | 10 Dec. 2019 | 29 Mar. 2019 | Range | Unit |
| GOT/ALT | 46 | 36 | 0-45 | U/L |
| GPT/AST | 37 | 26 | 0-41 | U/L |
| Total Protein | 73 | 69 | 60-82 | g/L |
| Albumin | 46 | 42 | 35-50 | g/L |
| Globulin | 27 | 27 | 20-40 | g/L |
| Alkaline | 89 | 80 | 30-115 | U/L |
| Phosphatase | | | | |
| (ALP) | | | | |
| Total Bilirubin | 10 | 16 | 2-20 | umol/L |
| GGT | 20 | 19 | 0-70 | U/L |
| Triglyceride | 1.3 | 0.7 | 0.3-4.0 | mmol/L |
| Total | 4.9 | 4.7 | 3.6-7.3 | mmol/L |
| Cholesterol | | | | |

Case 3. Age 31, Female, Chief complaint: irregular
menstrual cycle , Treatment date: 29 Jun. 2019

| | Before | 0.5 month | | |
|---|---|---|---|---|
| | treatment Date: | post Date: | Normal | |
| Test/Date | 24 Jun. 2019 | 10 Jul. 2019 | Range | Unit |
| GOT/ALT | 35 | 22 | 0-45 | U/L |
| GPT/AST | 28 | 23 | 0-41 | U/L |
| Total Protein | 76 | 74 | 60-82 | g/L |
| Albumin | 46 | 44 | 35-50 | g/L |
| Globumin | 30 | 30 | 20-40 | g/L |
| Alkaline | 57 | 50 | 30-115 | U/L |
| Phosphatase | | | | |
| (ALP) | | | | |
| Total Bilirubin | 6 | 10 | 2-20 | umol/L |
| GGT | 50 | 32 | 0-70 | U/L |
| Triglyceride | 1.5 | 1.2 | 0.3-4.0 | mmol/L |
| Total | 6.5 | 6.2 | 3.6-7.3 | mmol/L |
| Cholesterol | | | | |

Case 4. Age 28, Male, Chief complaint: Chronic
Fatigue, Treatment date: 29 Mar. 2019

| | Before | 3.5 months | | |
|---|---|---|---|---|
| | treatment Date: | post Date: | Normal | |
| Test/Date | 22 Mar. 2019 | 4 Oct. 2019 | Range | Unit |
| GOT/ALT | 65 | 26 | 0-45 | U/L |
| GPT/AST | 69 | 20 | 0-41 | U/L |
| Alkaline | 326 | 180 | 30-115 | U/L |
| Phosphatase | | | | |
| (ALP) | | | | |
| Total Bilirubin | 6 | 10 | 2-20 | umol/L |

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for preparing a monocytes derived signaling cells mixture, comprising the steps of:
(a) centrifuging a peripheral blood from a subject to collect buffy coats and platelet-poor-plasma;
(b) mixing the platelet-poor-plasma with a cell nutrients medium to obtain a cell culture medium;
(c) culturing the buffy coats in the cell culture medium for 24-168 hrs; and
(d) collecting the cultured buffy coats and the cultured cell culture medium from the step (c) to obtain the monocytes derived signaling cells (MDSC) mixture.

2. The method of claim 1, wherein the subject is a human.

3. The method of claim 1, wherein the leukocytes count of the subject is between 0.5-30×10⁹ nucleated cells/L.

4. The method of claim 1, wherein the ingredients of the cell nutrients medium comprise amino acids, glucose, sodium di-H phosphate, calcium chloride, magnesium chloride, potassium acetate, sodium chloride and dihydrogen monoxide.

5. The method of claim 1, wherein the volume ratio of the platelet-poor-plasma and the cell nutrients medium is 10:1 to 1:1.

6. The method of claim 1, wherein the cell density of the buffy coats ranges from 2.8-100×10⁹ nucleated cells/L.

7. The method of claim 1, wherein the cell density of the MDSC mixture ranges from 0.35-10×10⁹ nucleated cells/L.

8. The method of claim 1, wherein the cell ratio of the MDSC mixture comprises 0.5-15% monocytes.

9. The method of claim 8, wherein the cell ratio of the monocytes comprises 75-99% CD16+ monocytes.

10. The method of claim 1, wherein the cell ratio of the MDSC mixture comprises 0.3-15% CD34+ fibrocytes.

11. The method of claim 1, wherein the cell ratio of the MDSC mixture comprises 2-6% CD34+ fibrocytes.

12. The method of claim 1, wherein the MDSC mixture comprises angiogenin, EGF, NAP-2, leptin, PDGF-BB, RANTE, Acrp 30, IL-6R, TIMP-2, MSP-a, MCP-1, LIGHT, SDF-1, IL-8, TPO, VEGF, BDNF, IL-10, IGF-1, TGF-b, NAP-2, SCF, ICAM-1 and uPAR.

13. A product of monocytes derived signaling cells (MDSC) mixture, wherein the cell ratio of the MDSC mixture comprises 25-80% granulocytes, 15-70% lymphocytes, 0.3-15% fibrocytes and 0.5-15% monocytes, wherein the cell ratio of the monocytes comprises 75-99% CD16+ monocytes.

14. The product of claim 13, wherein the cell density of the MDSC mixture ranges from 0.35-10×10⁹ nucleated cells/L.

15. The product of claim 13, wherein the fibrocytes in the MDSC mixture comprises CD34+ fibrocytes.

16. A use of a composition for preparing a pharmaceutical composition for improving abnormal liver function index, wherein the composition comprises a monocytes derived signaling cells (MDSC) mixture, wherein the cell ratio of the MDSC mixture comprises 25-80% granulocytes, 15-70% lymphocytes, 0.3-15% fibrocytes and 0.5-15% monocytes, wherein the cell ratio of the monocytes comprises 75-99% CD16+ monocytes.

17. The use of claim 16, wherein the liver function index comprises glutamic oxaloacetic transaminase (GOT), glutamic pyruvic transaminase (GPT), total protein, albumin, globulin, alkaline phosphatase (ALP), total bilirubin, gamma-glutamyl transferase (GGT), triglyceride and cholesterol.

18. The use of claim 16, wherein the administration of the pharmaceutical composition comprises intravenous administration.

19. The use of claim 16, wherein the cell density of the MDSC mixture ranges from 0.35-10×10⁹ nucleated cells/L.

20. The use of claim 16, wherein the fibrocytes in the MDSC mixture comprises CD34+ fibrocytes.
